(19) United States
(12) Patent Application Publication
(10) Pub. No.: US 2026/0364323 A1
(43) Pub. Date: Nov. 19, 2026

(54) EMPTYING MONITORING SYSTEM FOR GASTROINTESTINAL TRACT DEVICES

(71) Applicant: TEVA PHARMACEUTICAL INDUSTRIES LTD., Tel Aviv (IL)

(72) Inventors: Avshalom BEN MENACHEM, Zur Izhak (IL); Lian ZALIT, Rosh Haain (IL)

(21) Appl. No.: 19/141,068

(22) PCT Filed: Dec. 21, 2023
(86) PCT No.: PCT/IB2023/063144

§ 371 Date: Jun. 19, 2025

Related U.S. Application Data
(60) Provisional application No. 63/434,449, filed on Dec. 21, 2022.

(51) Int. Cl. A61M 31/00

(52) U.S. Cl. CPC A61M 31/002; A61M 2205/0238; A61M 2205/3386; A61M 2210/1064

(57) ABSTRACT

Disclosed herein are apparatus, system, and method aspects for monitoring emptying of a retentive device from a predetermined location of a gastrointestinal tract. An aspect includes an expandable platform (100) used to dispense a medication at the predetermined location of the gastrointestinal tract that includes an emptying monitoring indicator that provides information on emptying of the expandable platform from the predetermined location. The expandable platform may include at least two articulating arms (102-n), a first and a second coupling head (104), a deformable member (106), and a locking assembly (212) that together allow the expandable platform to exist among various configurations. Additional aspects include a receiver (502) and/or a mobile device (504) for receiving a signal from the sensor and providing patient instructions accordingly.

### CROSS REFERENCE TO RELATED APPLICATIONS

This application claims the benefit of U.S. Provisional Patent App. No. 63/434,449, filed Dec. 21, 2022, the disclosure of which is hereby incorporated by reference herein.

### TECHNICAL FIELD

Aspects of the present disclosure relate to components, systems, and methods for monitoring emptying of retentive devices from a predetermined location of a gastrointestinal (GI) tract after temporary residence at the predetermined location.

### BACKGROUND

The gastrointestinal (GI) tract provides a therapeutic medium for an individual's body. At times, medications may need to be dispensed over a specified period of time within a predetermined location of the GI tract, such as within the stomach, the small intestine, or the large intestine, to treat symptoms of some medical conditions. Such medication dispensing can be accomplished by a device or mechanism that is retained at the predetermined location of the GI tract for the specified period of time (e.g., a retentive device) in order to dispense the medications or examine the region for the specified period of time. For example, such a device or mechanism can be temporarily retained by the pyloric valve, which controls material flow from the stomach, or the ileocecal valve, which controls material flow from the small intestine to the large intestine.

With some medical conditions, treatment requires administration of reoccurring, extended release doses of medications to a predetermined location of the GI tract. For example, a first dose of a medication dispensed through a first retentive device may be administered to the stomach on a first day, and a second dose of a medication dispensed through a second retentive device may be administered to the stomach on a later day. In this example, the first retentive device is designed to be retained in the stomach for a period of time in order to dispense the medications for this specified period of time. For safe and effective treatment, the first retentive device needs to empty out of the predetermined location of the GI tract (e.g., the stomach or small intestines) in order to make room for the second retentive device. There are certain mechanisms, such as internal timers (e.g., trigger assemblies), that can be built into retentive devices to allow them to empty out of the predetermined location of the GI tract after the specified period of time. But these mechanisms do not provide actual notice of the emptying.

### SUMMARY

In aspects presented herein, retentive devices, monitoring devices, monitoring systems, and monitoring methods provide for monitoring emptying of a retentive device from a predetermined location of a gastrointestinal (GI) tract after temporary residence at the predetermined location, where the retentive device was used to dispense a medication for a specified period of time to the predetermined location of the GI tract.

In an aspect, an example apparatus is an expandable platform used to dispense a medication to a predetermined location of a GI tract and an emptying monitoring indicator that provides information on emptying of the expandable platform from the predetermined location of the GI tract. With this aspect, the expandable platform includes at least two articulating arms, a first and a second coupling head, a deformable member, and a locking assembly that together allows the expandable platform to exist in a closed, expanded, or emptying configuration. Further in this aspect, the emptying monitoring indicator is coupled to the at least two articulating arms of the expandable platform. The emptying monitoring indicator may be a liquid-activated sensor having an erodible coating on an outer surface, or a magnet that emits a magnetic field.

In another aspect, an example system for detecting information from an emptying monitoring indicator is described. The example system includes a receiver that detects information from a retentive device, where the retentive device may be the example apparatus of the expandable platform used to dispense a medication to a predetermined location of a GI tract and the emptying monitoring indicator that provides information on emptying of the expandable platform from the predetermined location of the GI tract. Regardless, the information detected by the receiver indicates when the retentive device has emptied from the predetermined location of the GI tract. The example system also includes a mobile device that receives a signal from the receiver, based on the information detected from the retentive device. This signal may indicate whether it is safe for a patient to take another dose of the medication.

In yet another aspect, an example method for monitoring emptying of a retentive device from a predetermined location of a gastrointestinal tract is described. The method begins by ingesting the retentive device having an expandable platform for dispensing a medication and an emptying monitoring indicator coupled to the expandable platform. The method continues by positioning a receiver external to the body of the patient. The receiver detects information from the emptying monitoring indicator of the retentive device, and may be the receiver found in the example system. The method concludes by following patient instructions on a mobile device. The instructions are generated from a signal sent from the receiver based on the information detected by the receiver, and instruct a patient whether it is safe to take another dose of the medication. The mobile device may be the mobile device found in the example system.

Further features and advantages, as well as the structure and operation of various aspects, are described in detail below with reference to the accompanying drawings. It is noted that the specific aspects described herein are not intended to be limiting. Such aspects are presented herein for illustrative purposes only. Additional aspects will be apparent to persons skilled in the relevant art(s) based on the teachings contained herein.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the pertinent art to make and use the disclosure.

FIGS. 1A-1C are illustrations of an apparatus for monitoring emptying of a retentive device from a predetermined location of a gastrointestinal tract, according to some aspects of the present disclosure.

FIGS. 2A-2B are cross-sectional illustrations of an apparatus for monitoring emptying of a retentive device from a predetermined location of a gastrointestinal tract according to a first embodiment, in a closed configuration according to some aspects of the present disclosure.

FIGS. 2C-2D are cross-sectional illustrations of an apparatus for monitoring emptying of a retentive device from a predetermined location of a gastrointestinal tract according to a second embodiment, in a closed configuration according to some aspects of the present disclosure.

FIGS. 3A-3B are cross-sectional illustrations of an apparatus for monitoring emptying of a retentive device from a predetermined location of a gastrointestinal tract, in an expanded configuration according to some aspects of the present disclosure.

FIG. 4 is an illustration of an apparatus for monitoring emptying of a retentive device from a predetermined location of a gastrointestinal tract, in an emptying configuration according to some aspects of the present disclosure.

FIGS. 5A-5B are illustrations of a system for detecting information from an emptying monitoring indicator, according to some aspects of the present disclosure.

FIG. 6 is a flowchart of a method for monitoring emptying of a retentive device from a predetermined location of a gastrointestinal tract, according to some aspects of the present disclosure.

FIG. 7 is a flowchart of a method for monitoring emptying of a retentive device from a predetermined location of a gastrointestinal tract, according to some aspects of the present disclosure.

FIG. 8 is a block diagram of an example computer system useful for implementing various aspects of the present disclosure.

In the drawings, like reference numbers generally indicate identical or similar elements. Additionally, generally, the left-most digit(s) of a reference number identifies the drawing in which the reference number first appears.

Aspects of the present disclosure will be described with reference to the accompanying drawings.

### DETAILED DESCRIPTION

Provided herein are system, apparatus, device, method and/or computer-readable medium aspects, and/or combinations and sub-combinations thereof for monitoring emptying of a retentive device from a predetermined location of a gastrointestinal (GI) tract after temporary residence at the predetermined location.

As used herein, the term “predetermined location” may be used to refer a particular location or a region along the GI tract. In some aspects, the predetermined location may be used to refer to a region consisting of the stomach, and for example including at the pyloric valve. In other examples, the predetermined location may be at the ileocecal valve region and may be used to refer to a region generally including one or more of the lower part of the small intestine (terminal ileum), the ileocecal valve, the cecum and the ascending colon.

Several aspects rely on environmental conditions of the GI tract. Environmental conditions may refer to biological, physical, or chemical conditions of an environment in which the device is positioned or intended to be positioned. The environmental conditions may include for example, temperature, pH, atmospheric pressure, gravity, electromagnetic field, vibration, glucose concentration, oxygen concentration, enzyme concentration, etc. In a predetermined location of the GI tract, for example the stomach or pyloric region, or the ileocecal valve region, standard environmental conditions may refer to average physiological conditions observed in said part of the GI tract. For example, standard pH conditions for different parts of the human GI tract are summarized in the table below:

| | pH along the | | |
|---|---|---|---|
| | gastrointestinal tract (1) | | |
| | | 25^{th} | 75^{th} |
| GI segment | median | percentile | percentile |
| stomach | 1.5 | 1.1 | 1.8 |
| duodenum | 6.4 | 5.7 | 6.9 |
| proximal small intestine | 6.6 | 6.2 | 7.1 |
| mid small intestine | 7.1 | 6.6 | 7.4 |
| distal small intestine (incl. ICV) | 7.3 | 7.0 | 7.5 |
| cecum | 5.7 | 5.2 | 6.3 |
| ascending colon | 5.6 | 5.1 | 6.5 |
| transverse colon | 5.8 | 5.3 | 6.4 |
| descending colon | 6.6 | 6.3 | 7.0 |
| rectum | 6.6 | 5.8 | 7.1 |

The GI tract is a series of hollow organs of the digestive system that leads from the mouth to the anus, including the mouth, esophagus, stomach, small intestine, large intestine, and anus. The small intestine includes the duodenum, jejunum, and ileum. And the large intestine includes the appendix, cecum, colon, and rectum. Dispensing medications within a particular location of the GI tract inside a body for a sufficiently long period of time may be useful for treating medical conditions, for example, by dispensing a medication (e.g., a topical treatment) directly in the vicinity of the particular location or for improving action of medications which have an improved absorption into the blood circulation, or by releasing an amount of the medication consistently over a prolonged period of time (hours, days, weeks, etc.). Medications can be dispensed in such a manner using a retentive device, a device that is retained at a predetermined location of the GI tract for a specified period of time.

Retentive devices for medication administration may be particularly useful for the dispensing medications that are preferentially absorbed in the stomach, have better solubility in the stomach, are intended for local treatment of the stomach or duodenum, and/or are poorly soluble in alkaline pH or the intestines, as well as those medications that degrade in the colon or in the intestine, those medications that disturb colonic microbes, those medications with poor patient adherence, or medications that have slow and or incomplete intestinal absorption.

Retentive devices for medication administration through gastric release may be particularly useful in treating a gastric ulcer, gastroesophageal reflux disease, stomach cancer, or stomach infections. Retentive devices for medication administration through ileac and colonic release may be particularly useful in treating Inflammatory Bowel Disease (IBD) such as Ulcerative Colitis and Crohn's disease, colonic cancer, or colonic infections or GI site specific conditions such as Immunoglobulin A Nephropathy which involves generation of pathogenic entities with terminal ileum gut-associated lymphoid tissue Peyer's Patches.

When using retentive devices for medication administration, the retentive device may exist in different configurations, including a configuration that allows it to be ingested by a patient. Additionally, the different configurations may include a configuration that allows the retentive device to remain at a predetermined location of the GI tract for a specified period of time, as well as a configuration that allows the retentive device to exit the predetermined location of the GI tract after the specified period of time. Certain mechanisms, such as internal timers (e.g., trigger assemblies), can be built into retentive devices to allow them to empty out of the predetermined location of the GI tract after the specified period of time. For certain configurations of retentive devices it is important for the retentive devices to be emptied/eliminated from the GI tract, especially if another dose of a medication needs to be dispensed through another retentive device. Without such emptying, multiple retentive devices could clog the GI tract, blocking proper flow of fluids and materials (e.g., chyme). Further, it may be dangerous for a patient to ingest a second dose of a drug before the first dose has been completely delivered or has emptied out of the gastrointestinal tract. A design and planning of such device dosed in a repeated manner (e.g. once daily, once weekly) may encounter risk of accumulation in the GI tract. Several lines of defense may be implemented to mitigate this risk such as incorporating in device elements made of materials which cannot resist GI conditions for more than twice the time in which the device was planned to stay in the GI, or applying a technique of external triggering to activate device emptying before giving the next device. An additional line of defense that should be considered is the emptying monitoring indicator described herein, which can be incorporated in the device so to notify that a first platform is emptied before the second one is dosed. Current apparatuses, systems, and methods of retentive devices do not provide actual notice of the emptying, making it difficult to determine when another dose of a medication can be safely and effectively administered.

Aspects herein are aimed to solve this technological problem using an innovative emptying monitoring indicator coupled to a retentive device (i.e., an expandable platform). In some aspects, the emptying monitoring indicator may be a liquid-activated sensor that is enclosed in an erodible coating or a magnet that emits a magnetic field. The emptying monitoring indicator may interact with a system that sends and receives signals and provides patient instructions. For example, the retentive device with the coupled emptying monitoring indicator may expand at a predetermined location of the GI tract for a specified period of time, dispensing medication. When the retentive device with the coupled emptying monitoring indicator has dispensed the medication for the specified period of time, it will begin emptying from the predetermined location of the GI tract. In one example, the erodible coating enclosing the liquid-activated sensor may erode based on certain conditions of the GI tract, such as pH or other GI environmental features (e.g., prolonged exposure to colonic enzymes), exposing the liquid-activated sensor to GI tract fluids. Based on the configuration of the erodible coating and the activation of the sensor, signals are sent and/or received, and it is confirmed whether the retentive device with the coupled emptying monitoring indicator has emptied out of the predetermined location of the GI tract. Patient instructions may be generated from those signals, for example instructing a patient that it is safe to take another dose of medications through another retentive device.

Aspects herein provide various benefits. For example, the emptying monitoring indicator provides better safety through confirmation of emptying from a predetermined location of the GI tract. In other words, it can be known for certain whether a retentive device remains at the predetermined location, or whether it has begun the process of emptying out of the GI tract. Additionally, the emptying monitoring indicator can provide more effective treatment through information on changes in emptying time or more specific medication dosing methods. In other words, the time between ingesting the retentive device with the coupled emptying monitoring indicator and notification of when the device has emptied from a predetermined location of the GI tract can be used in analyzing patient status and providing further treatment. Therefore, the innovative approach of using an emptying monitoring indicator coupled to a retentive device (i.e., an expandable platform) solves the above technological problem by providing actual notice of when the device has emptied from the predetermined location of the GI tract.

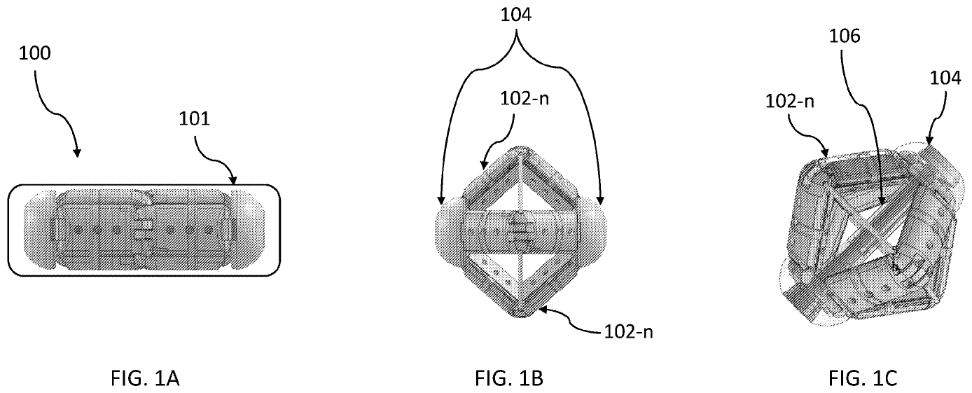

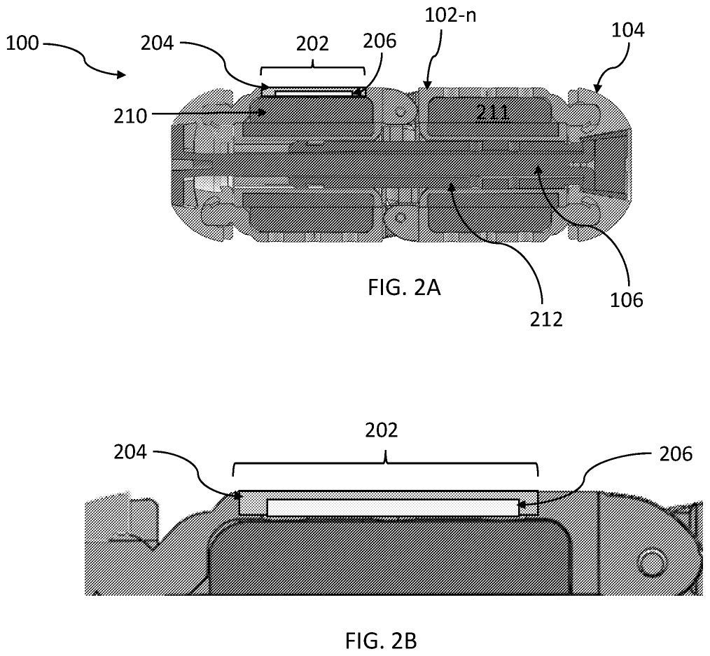

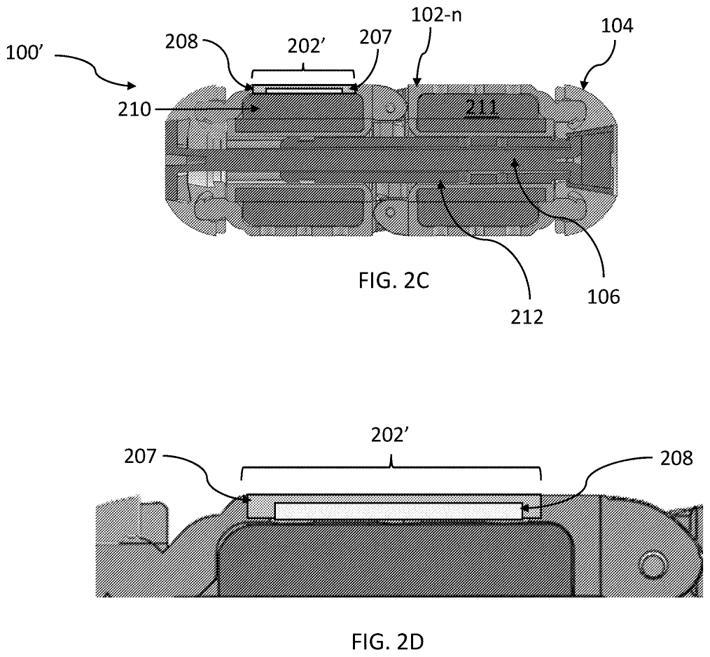

FIGS. 1A-1C are illustrations of an expandable platform 100 for monitoring emptying of a retentive device from a predetermined location of a gastrointestinal tract, according to some aspects of the present disclosure. FIG. 1A illustrates expandable platform 100 in a closed configuration. FIGS. 1B and 1C illustrate expandable platform 100 in an expanded configuration, where FIG. 1B shows a front view of expandable platform 100 and FIG. 1C shows an isometric view of expandable platform 100. Expandable platform 100 is further described with reference to FIGS. 2A-2D, FIGS. 3A-3B, and FIG. 4. Though one embodiment of an expandable platform 100 is shown, it is contemplated that the emptying monitoring indicator 202 as described herein can be utilized with other expandable platform embodiments, such as the expandable platforms described in PCT Publication No. WO 2021/038572, PCT Publication No. WO 2022/043948, PCT Publication No. WO 2022/043950, and PCT Publication No. WO 2022/185220, the disclosures of each of which are incorporated by reference herein in their entirety.

Expandable platform 100 includes an emptying monitoring indicator. The expandable platform 100 can include a capsule 101 surrounding portions of the expandable platform 100 and configured to maintain the expandable platform 100 in the closed configuration until the expandable platform 100 reaches the intended gastric residence location. The capsule 101 can comprise a material configured to erode under predetermined gastric conditions, where the material can comprise. In some aspects, the container may be at least partially made of a material dissolving in the environmental conditions of the predetermined location while not dissolving or having a slower dissolving rate in the esophagus so to enable safe swallowing. The container may be made of or coated for example with Eudragit® EPO that dissolves in pH<5 in the stomach use of the device, or made of an enteric polymer in the ICV use of expandable platform 100. For example, expandable platform 100 in the closed configuration may be temporarily maintained in the closed configuration by a capsule as a container.

The expandable platform 100 is a retentive device and includes two articulating arms 102-n, first and a second coupling heads 104, and a deformable member 106. In embodiments, the expandable platform 100 can include more than two articulating arms 102-n. Each of FIGS. 2A and 2B illustrate a cross-sectional view of expandable platform 100 in a closed configuration, showing the internals of expandable platform 100 according to aspects of a first embodiment the present disclosure. FIGS. 2C and 2D illustrate a cross-sectional view of expandable platform 100 in a closed configuration, showing the internals of expandable platform 100 according to aspects of a second embodiment the present disclosure. With reference to FIGS. 2A-2D, the expandable platform 100 includes locking assembly 212 and a medication cavity 210 containing a medication 211. With further reference to FIGS. 2A-2B, expandable platform 100 includes emptying monitoring indicator 202. With further reference to FIGS. 2C-2D, expandable platform 100′ includes emptying monitoring indicator 202′. FIGS. 2A-2B illustrates an aspect of expandable platform 100 in a closed configuration and with emptying monitoring indicator 202 including liquid-activated sensor 206 and erodible coating 204. FIGS. 2C-2D illustrates an aspect of expandable platform 100′ in a closed configuration and with emptying monitoring indicator 202′ including magnet 208.

Expandable platform 100 is configured to dispense a medication 211 at a predetermined location of the GI tract through temporary residence at the predetermined location. The predetermined location may be the stomach or any location within the small intestine or the large intestine.

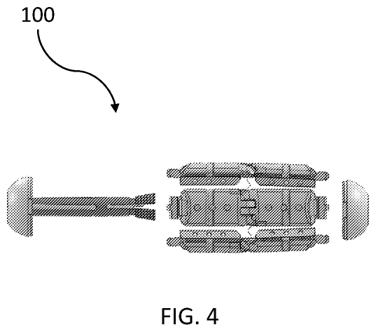

The expandable platform is configured to transfer among various configurations. These configurations may include closed, expanded, and emptying configurations. The closed configuration of expandable platform 100 may be the state of expandable platform 100 prior to administration, where expandable platform 100 has a size that is suitable for swallowing or for manipulation with an endoscope. For example, the expandable platform may be configured to be orally ingested in a closed configuration (see FIG. 1A). The closed configuration may have a compact shape to facilitate frequent administration of expandable platform 100 through ingestion. Thus, the expandable platform in the closed configuration may be capable of being fitted into a cylinder of about 35 mm length or smaller and of about 12 mm diameter or smaller, for example in a cylinder of 30 mm in length and of 10 mm or 9 mm in diameter. The expandable platform is configured to transfer from the closed configuration into an expanded configuration (see FIGS. 1B and 1C). The expanded configuration of expandable platform 100 may be the state of expandable platform 100 when it resides at the predetermined location. In addition, when the expandable platform is in the expanded configuration and positioned at the predetermined location of the GI tract, the expandable platform is configured to be retained at the predetermined location of the gastrointestinal tract. The dimensions of expandable platform 100 in the expanded configuration may be such that it can be fitted in a sphere of a diameter of about 35 mm or less, and preferably of about 30 mm or 25 mm or less. Thus, the dimensions of the expandable platform in the expanded configuration may be such that the device cannot pass through an orifice of between 15 millimeters and 20 millimeters diameter. The expandable platform may be further configured to irreversibly transfer from the expanded configuration into an emptying configuration (see FIG. 4). The emptying configuration may be a disassembled configuration. The expandable platform is configured to move past the predetermined location of the gastrointestinal tract in the emptying configuration, so as to be emptied from the GI tract of the patient.

Because the predetermined location may be the stomach or any location within the small intestine or the large intestine, the expandable platform may be configured in the expanded configuration to be retained in the stomach of the patient, and to pass through the pyloric valve in the emptying configuration. In another aspect, the expandable platform may be configured in the expanded configuration to be retained at the ileocecal valve of the patient, and to pass through the ileocecal valve in the emptying configuration. In either case, the expandable platform is configured to allow chyme flow while the expandable platform is in the expanded configuration, positioned and retained at the predetermined location of the gastrointestinal tract. In other words, chyme is allowed to flow through or by expandable platform 100.

The expandable platform transfers among various configurations through interactions of its various components. These components are described independently, and in relation to one another.

At least two articulating arms 102-n may be composed of one or more arm sections. At least two articulating arms 102-n may be an elongated structure that includes at least one articulation (e.g., a joint) between two arm sections, allowing bending during which the two arm sections substantially maintain their shape. The respective arm sections of the at least two articulating arms 102-n may be coupled end to end with a pivot-type coupling.

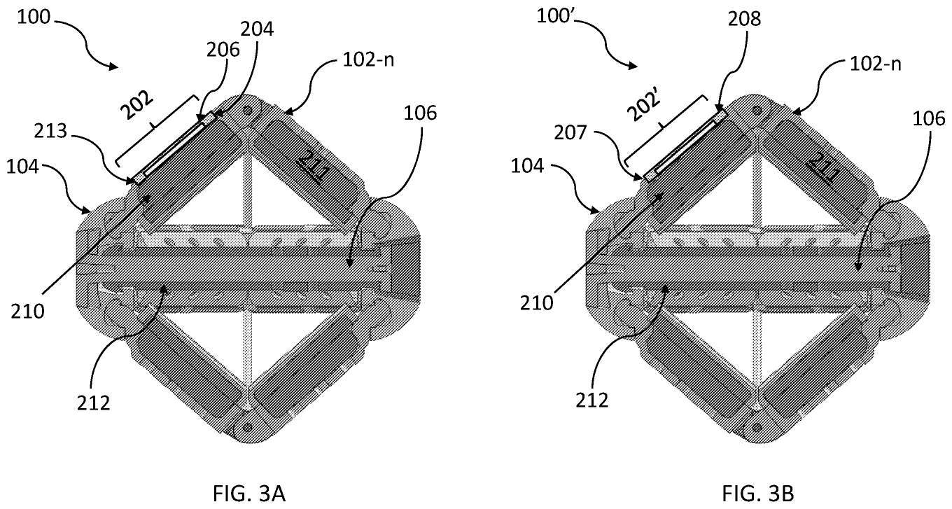

At least one of the at least two articulating arms 102-n may have one or more cavities, defined by the surfaces of the at least two articulating arms 102-n. The one or more cavities may be partially exposed to the environment of the GI tract. For example, at least one of the at least two articulating arms 102-n may have a medication cavity 210 for storing and dispensing medications. Additionally, at least one of the at least two articulating arms 102-n may have an indicator cavity 213 for coupling an emptying monitoring indicator 202. As depicted in FIG. 3A, one of the medication cavities 210 and the indicator cavity 213 may be adjacent to each other. However, it is contemplated that the medication cavities 210 and the indicator cavity 213 can be alternatively positioned as desired.

Each of the at least two articulating arms 102-n may be made of any rigid material, such as plastic (e.g., a resin), metal, or ceramic, and may be 3D printed or (e.g., injection) molded. Each of the at least two articulating arms 102-n may be made of a biocompatible material. The biocompatible material may optionally be biodegradable, and further optionally be configured for being retained at the predetermined location for a specified period of time and for a partial or complete erosion before emptying from the body. For example, each of the at least two articulating arms 102-n may be made of MEDIC® rigid material manufactured by Stratasys® 3D printer or Visijet M3 Crystal 3D printer by Objet® or for example made of cellulose acetate or other biocompatible and biodegradable relevant polymers manufactured by molding. The rigidity of at least two articulating arms 102-n may provide high structural rigidity and retention capability of the device (capacity of the device to be retained), while achieving a particularly high ratio of expanded volume relative to compactness of the closed configuration, due to allowed thinness of the arms. Each of the at least two articulating arms 102-n may be made in different materials from one another or the same materials. Each of the at least two articulating arms 102-n may be structurally similar and of the same shape. Each of the at least two articulating arms 102-n may be straight. Each of the at least two articulating arms 102-n may be arranged in one or more layers in the expandable platform. Each of the at least two articulating arms 102-n has a first end and a second end.

First and second coupling heads 104 may be identical in their external shape, each substantially centered on the longitudinal axis, or arranged symmetrically relative to one another with respect to a median plane of expandable platform 100, perpendicular to the longitudinal axis. Each coupling head 104 may be integrally formed or made of any material (identical to or different from at least two articulating arms 102-n), for example a rigid material, such as plastic (e.g., a resin), metal, or ceramic, or a semi-rigid or articulating material. Each coupling head 104 may be 3D printed or (e.g., injection) molded. Each coupling head 104 may be made of a biocompatible material. The biocompatible material may optionally be biodegradable, and further optionally be configured for being retained at the predetermined location for a predetermined time and for a partial or complete erosion before emptying from the body. For example, each coupling head 104 may be made of MEDIC® rigid material manufactured by Stratasys® 3D printer or Visijet M3 Crystal 3D printer by Objet® or for example made of cellulose acetate manufactured by molding. The rigidity of the coupling heads 104 may provide high structural rigidity of expandable platform 100.

In both the closed configuration (see FIG. 1A) and the expanded configuration (see FIGS. 1B and 1C), the first end of each of the at least two articulating arms 102-n may be coupled to first coupling head 104, and the second end of each of the at least two articulating arms 102-n may be coupled to second coupling head 104. Thus, at least two articulating arms 102-n are configured to join to each other by both ends, such that the arms are secured alongside each other in both the closed configuration and the expanded configuration.

The first end of each of the at least two articulating arms 102-n may be rotatably coupled to the first coupling head 104, and the second end of each of the at least two articulating arms 102-n may be rotatably coupled to the second coupling head 104. Expandable platform 100 may thus form a (e.g., rigid) mobile (e.g., Sarrus) mechanism deformable from the closed configuration into the expanded configuration, optionally reversibly deformable between the expanded configuration into the closed configuration, by converting relative rotations into a relative motion (e.g., a translation along axis X) between the first and the second coupling head 104. The converted relative rotations are the relative rotation of each of the coupled at least two articulating arms 102-n, one with respect to the other, and the relative rotation of each of at least two articulating arms 102-n coupled to one of the first or second coupling head 104. Such motion conversion achieves bending of each of at least two articulating arms 102-n to deform expandable platform 100 from the closed configuration into the expanded configuration. In some aspects, the at least two articulating arms 102-n are bent in the expanded configuration, whereas the at least two articulating arms 102-n are substantially straight in the closed configuration. In examples, due to their rigidity, the arm sections of the at least two articulating arms 102-n maintain their shape during the whole motion conversion, and thereby each present a substantially identical shape between the expanded configuration and the closed configuration.

Expandable platform 100 may be a prism (e.g., cylindrical) shape in the closed configuration. Each arm section of the at least two articulating arms 102-n may maintain its shape (i.e., not be deformed) during use of expandable platform 100, for example during transfer from the closed configuration into the expanded configuration, or during transfer from the expanded configuration into the emptying configuration. Each arm section of the at least two articulating arms 102-n may be substantially of a same length, optionally structurally identical. The device may thus have a bipyramidal shape in the expanded configuration. Each arm section of the at least two articulating arms 102-n may correspond to an edge of the bipyramidal shape, while each pivot-type coupling and each coupling head 104 may correspond to a vertex of the bipyramidal shape. Expandable platform 100 is thus configured to switch the compact prism/straightened shape of the closed configuration (with a relatively small volume occupancy) into the generally hollow bipyramidal shape of the expanded configuration (with a relatively large volume occupancy). Components of expandable platform 100 may be rounded at edges or vertices of the bipyramidal (e.g., octahedral) shape, such that expandable platform 100 may present in the expanded configuration a generally spherical convex hull 3D structure.

Specifically, the deformable member 106 may be configured to cause the expandable platform 100 to transition from the closed configuration to the expanded configuration. When the expandable platform 100 is in the closed configuration, the deformable member 106 may be under tension, but prevented from causing the expandable platform 100 from transitioning to the expanded configuration by the capsule 101. Following oral administration, the capsule 101 may dissolve at or before the predetermined location thereby releasing expandable platform 100 into the expanded configuration. In examples where the predetermined location is the ICV, following oral administration, the capsule 101 (e.g., an enteric coated capsule 101) may dissolve in the small intestine thereby releasing the device into the expanded configuration. Once the capsule 101 sufficiently erodes, tension imparted by the by the deformable member 106 may cause the deformable member 106 to contract, thus rotating the at least two articulating arms 102-n and drawing the coupling heads 104 linearly towards each other to transition the expandable platform 100 from the closed configuration to the expanded configuration.

Referencing FIG. 2A, may be configured to hold expandable platform 100 in the expanded configuration. The expandable platform thereby maintains dimensions preventing it from moving past the predetermined location of the GI tract. Locking assembly 212 may be made of a more rigid or less elastic material than deformable member 106. Locking assembly 212 may present a higher tensile strength, a higher tensile modulus, or a lower elongation percentage at break, compared to deformable member 106. For example, locking assembly 212 may be made of a rigid or semi-rigid material, such as cellulose acetate, Polyether ether ketone (PEEK), or polyurethane, or other biocompatible materials such as polylactic (PLA), polyglycolic (PLG) polymers.

Locking assembly 212 may be shock-absorbing. This reduces risks of damage to the body tissue of the GI tract, by providing some resilience to expandable platform 100. The material composition or the structure design of the locking member may further convey a shock absorption capability to expandable platform 100 when exposed to GI forces in the expanded configuration. Such shock absorption capability makes expandable platform 100 more compliant to GI tissue.

Locking assembly 212 may interact with the first and second coupling heads 104 and deformable member 106 in order to maintain the expanded configuration. A first end of each of the at least two articulating arms 102-n may be coupled to the first coupling head 104 and a second end of each of the at least two articulating arms 102-n may be coupled to the second coupling head 104. Deformable member 106 may be arranged between the at least two articulating arms 102-n and configured to force the coupling heads 104 together to bend the at least two articulating arms 102-n, thereby forcing the device into the expanded configuration. Thus, first and second coupling heads 104 come close one to another in the expanded configuration due to retraction of the deformable member 106. Locking assembly 212 may be arranged longitudinally between coupling heads 104 or along deformable member 106. In some aspects, locking assembly 212 may surround at least a (e.g., longitudinal) portion of deformable member 106, at least partially on the periphery of said portion. This allows compactness of expandable platform 100.

Locking assembly 212 may be activated by snapping (i.e., snap-fitting) one or more parts of the locking assembly 212 with a cooperating component of expandable platform 100. The snapping or snap-fitting may for example comprise one or more cantilever snap-fits. Alternatively, locking assembly 212 may be activated by a latching mechanism. For example, one or more parts of locking assembly 212 may each engage with a cooperating component of expandable platform 100, and a latch may be actuated upon the engagement and fixedly attach the cooperating component.

In some aspects, locking assembly 212 may comprise a locking member arranged between coupling heads 104 and having two ends. At least one end of the locking member is free in the closed configuration. In other words, said at least one end is free or unattached from a respective coupling head 104 with which said at least one end is to cooperate. Yet in other words, a distance separates said at least one end and its respective coupling head 104, such that they are disconnected. Further, the locking member is configured for each such free end to engage its respective coupling head 104 when the device transfers from the closed configuration into the expanded configuration, thereby attaching the end to its respective coupling head 104. This allows to efficiently make use of the force used to pull the two coupling heads together to perform the locking. The case of only one free end for the locking member facilitates activation of the locking assembly.

In other aspects, the respective coupling head 104 of at least one free end of the locking member may comprise a recess configured for receiving the free end of the locking member and attaching said end to said respective coupling head 104 when expandable platform 100 transfers from the closed configuration into the expanded configuration. For instance, the recess may be configured for snapping the free end of the locking member therein. This facilitates the locking of the device in the expanded configuration. Also, the snapping (e.g., cantilever snap-fit) may lock the free end of the locking member, meaning that the force necessary to unsnap the locking member is higher (e.g., significantly, e.g., at least twice or three times higher) than the force necessary to perform the snapping. The snapping may be effective due to deformable member 106 pulling the two coupling heads 104 together at a sufficient speed or with a sufficient force.

At least one free end of the locking member may have an anchor shape and is configured to be forced into its respective coupling head 104, coupling head 104 being configured for retaining the free end afterwards. By “anchor shape”, it is meant any generally longitudinal shape with one or more radial protrusions at its extremity, for example a peripheral radial protrusion or one or more radial protrusions peripherally distributed. For instance, such an anchor shape may be formed by locking pins or locking dents protruding radially from the locking member. In turn, each coupling head 104 configured for receiving in a recess such an anchor shape may comprise a corresponding shape in the recess cooperating with the radial protrusions of the anchor shape, for example radial recesses or protrusion, to achieve snap-fitting. The anchor shape allows that the force necessary to perform the locking of the locking member's free end(s) in the respective coupling head(s) 104 be lower than the force necessary to unlock said free end(s). In other words, the anchor shape enables snapping. In addition, at least one (e.g., each) free end of the locking member may have a rounded extremity so as to be more easily forced into its respective coupling head 104. In addition, at least one (e.g., each) free end of the locking member or the receiving recess of the respective coupling head 104 may be deformable so as to be reduced in diameter (i.e., dimension across a longitudinal snapping axis) during the snapping, so as to facilitate the snapping. The anchor shape or the shape of the receiving recess may be such that the deformation only occurs in one direction along the snapping axis, i.e., in the snapping direction, while in the opposite direction, the anchor shape and the recess cooperate such that one retains the other, for example thanks to retaining edges (e.g., generally orthogonal to the snapping axis).

In particular, the unfolding force, that is, the force necessary to expand expandable platform 100 and perform the locking (e.g., >250 gr) (i.e., activate the locking assembly) may be lower (e.g., significantly, for example at least twice or at least three times) than the emptying force, that is, the force necessary to perform unlocking (e.g., 400-500 gr) (i.e., deactivate/release the locking assembly) and compact the device. As a result, deformable member 106 may be optimized for its main function, that is, the expanding function (also referred to as “unfolding” function), while the locking assembly may take over when it comes to the retention function.

Deformable member 106 pulls first and second coupling heads 104 together so as to bend at least two articulating arms 102-n and thereby impart the expanded configuration to expandable platform 100 from the closed configuration. For that, the resiliently deformable member presents a certain tensile strength, while displaying resilience capacity, and is made of a certain type and quantity of material. Deformable member 106 may be in the form of a tube or a spring (e.g., helical spring). Deformable member 106 may be configured to be stretched (along the longitudinal axis, into the closed configuration). For example, deformable member 106 may be elastic (i.e., made of an elastic material, such as silicone). For example, deformable member 106 may be made of silicone shore 55A, for example as a section of a silicone tube having a cross section area of about 3 mm². Alternatively, deformable member 106 may be made of metal, such as stainless steel or Nickel titanium. Deformable member 106 may be arranged between the arms. Deformable member 106 may be secured to both the first and second coupling heads 104.

Deformable member 106 is connectable to first and second coupling heads 104. Deformable member 106 has two ends, each configured to pull a respective coupling head 104. In some aspects, at least one end of deformable member 106 is free in the closed configuration. In other words, at least one end is free or unattached from a respective coupling head 104 with which said at least one end is to cooperate; a distance separates said at least one end and its respective coupling head 104, such that they are disconnected. Thus, deformable member 106 is relaxed, that is, not fully stretched (i.e., at least to a lesser point than if it were attached to the coupling heads 104 on both ends). Expandable platform 100 is configured for transferring deformable member 106 from a relaxed state into a strained state by imparting a certain amount of straining to the deformable member 106. This straining may also be referred to as “cocking”, as it amounts to supply it with potential energy. The stretching of deformable member 106 may be performed while expandable platform 100 stays in the closed configuration, thus while maintaining locking assembly 212 as inactive (e.g., keeping the locking member's one or two free ends unattached from the respective one or two coupling heads 104). Locking assembly 212 may thus be inactive even when deformable member 106 is in the strained state.

As illustrated in FIGS. 3A and 3B, first and second coupling heads 104 may come close to one another in the expanded configuration due to retraction of deformable member 106. Specifically, deformable member 106 forces first and second coupling heads 104 to move towards each other during transfer of the expandable platform from the closed configuration into the expanded configuration, such that first and second coupling heads 104 are closer to one another in the expanded configuration than in the closed configuration. Such deformation is allowed by at least two articulating arms 102-n, which further impart expansion to the device, due to their bending. The expandable platform may further comprise a passage between the articulating arms when the articulating arms are bent, so as to allow chyme flow in the expanded configuration.

Medication cavity 210 is configured to dispense a medication 211 while allowing chyme flow, when expandable platform 100 is positioned at the predetermined location and in the expanded configuration. Medication cavity 210 may be defined by the structure of at least one of the at least two articulating arms 102-n. In particular, medication cavity 210 may be an opening within the center of at least one of the arm sections of the at least two articulating arms 102-n. In some aspects, medication cavity 210 may comprise a peripheral wall, the peripheral wall having apertures formed thereon, the apertures providing exposure of medication cavity 210. In alternative aspects, medication cavity 210 may comprise an exposed recess, the exposed recess lodging at least part of the load of the medication 211. In further alternative aspects, medication cavity 210 may comprise a coating on an exposed surface of expandable platform 100, the coating containing at least part of the medication.

The medication may be solid or contain one or more pharmaceutically acceptable excipients. The solid medication may comprise a therapeutic payload for local release or topical treatment of a patient condition at the gastric region or for systemic absorption. The solid medication may comprise no protective cover (e.g., a coating), as expandable platform 100 may comprise a container for delivery at the predetermined location, thereby ensuring that the medication remains functional until it reaches the predetermined location. Alternatively, the solid medication may have a coating, so as to dispense the medication only when desired. In other aspects, the medication may be carried by expandable platform 100 in a solid, semi-solid, powder, gel, or liquid form.

Emptying monitoring indicator 202 may provide information used to determine when expandable platform 100 has emptied from the predetermined location of the GI tract. Referring to FIGS. 2A-2B, in one embodiment the emptying monitoring indicator 202 may be coupled to the expandable platform through at least one of the at least two articulating arms 102-n. Though FIGS. 2A-2B and 3A describe expandable platform 100 and FIGS. 2C-2D and 3B describe expandable platform 100′, like reference numbers will be used in both embodiments for similar components and the related description shall only be described once for brevity, and shall accordingly apply equally to each embodiment. In some aspects, emptying monitoring indicator 202 may be coupled to the expandable platform through an arm segment of at least one of the at least two articulating arms 102-n, such as through being received within an indicator cavity 213 of one of the at least two articulating arms 102-n. A portion of at least one of the at least two articulating arms 102-n or its arm segment may be constructed with an excised space (e.g., indicator cavity 213) to allow emptying monitoring indicator 202 to fit flush with an outer surface of at least one of the at least two articulating arms 102-n. Emptying monitoring indicator 202 may be coupled to the expandable platform via an adhesive, a welding or soldering connection, 3D printed or (e.g., injection) molded as part of the expandable platform, or any other type of coupling safely compatible with the GI tract. Alternatively, the liquid-activated sensor 206 can be placed within the medication cavity 210.

FIG. 3A is a cross-sectional illustration of an expandable platform 100 including the emptying monitoring indicator 202 for monitoring emptying of a retentive device from a predetermined location of a GI tract, in an expanded configuration according to some aspects of the present disclosure, while FIG. 3B is a cross-sectional illustration of an expandable platform 100′ including emptying monitoring indicator 202′. In some aspects, as illustrated in FIG. 3A, emptying monitoring indicator 202 may be a liquid-activated sensor 206 coupled to at least one of the at least two articulating arms 102-n of the expandable platform, through one of the previously described couplings. Liquid-activated sensor 206 may include a liquid level switch that is designed to send a signal when immersed in a liquid, such as water or chyme fluid. Alternatively, liquid-activated sensor 206 may include components that are altered by contact with water or chime fluid so as to initiate or alter sensor function. Liquid-activated sensor 206 may be an optical sensor, a capacitive sensor, a conductive sensor, a diaphragm sensor, a float sensor, or any other type of liquid-activated sensor that can be coupled to the expandable platform. In some aspects, liquid-activated sensor 206 is an electronic sensor requiring an energy source. The energy source may be a battery coupled to liquid-activated sensor 206, or another internal or external energy source. Such sensor principle could be embedded in the platform, sealed with a coating (e.g. the erodible coating 204) and or fixed within a polymer that delays the signal to a predefined timing.

In some aspects, liquid-activated sensor 206 is coupled to at least one of the at least two articulating arms 102-n or its arm segment through an interface. In other aspects, liquid-activated sensor 206 is coupled directly to at least one of the at least two articulating arms 102-n or its arm segment. Liquid-activated sensor 206 may be coupled to the at least one of the at least two articulating arms 102-n or its arm segment through the indicator cavity 213. This means that liquid-activated sensor 206 would have a coupled surface, and also an outer surface that may allow liquid-activated sensor 206 to directly contact the GI tract environment. The outer surface may be on the sides and/or top of liquid-activated sensor 206, depending on the dimensions of the liquid-activated sensor 206 and the dimensions of the indicator cavity 213. In some aspects, the outer surface of the liquid-activated sensor 206 may be enclosed by an erodible coating 204, which may be an erodible matrix, so that the outer surface is not initially in direct contact with the GI tract environment. Examples of erodible coatings 204 that can be utilized to isolate the liquid-activated sensor 206 from the surrounding gastric embodiment will be described further below.

In some aspects, erodible coating 204 may erode based on conditions of the organs of the GI tract (e.g., pH, enzymatically). For example, an expandable platform 100 with emptying monitoring indicator 202 may dispense medications in the stomach for a period of time. The environment of the stomach has a certain pH or microbial composition that may not be capable of eroding erodible coating 204. However, once the retentive device with the coupled emptying monitoring indicator 202 has dispensed the medication in the stomach for the period of time, it will close and empty from the stomach into the small intestine. The environment of the small intestine may have a certain pH or microbial composition different from that the environment of the stomach that is capable of eroding erodible coating 204. Similarly, the large intestine may have a certain pH or microbial composition different from that of the environment of the small intestine that is capable of eroding erodible coating 204. Alternatively, the erodible coating 204 may be configured to dissolve at the pH or under the microbial composition of the medication delivery site (i.e., stomach or small intestine) rather than under conditions of a gastric site subsequent to that of medication delivery, but over a period of time that corresponds to the intended period of retention of the expandable platform 100 at the predetermined location.

In one embodiment, the erodible coating 204 may be an enzymatically degredable polymer, whose degradability or dissolubility changes depending on conditions within the surrounding colonic environment (e.g., unique micro-flora/microbiota fermentation). For example, the erodible coating 204 may comprise a colonic erodible matrix, which responds to colonic conditions such as enzymes particular to the colon. Colonic microbes are almost not available in the stomach or small intestine so the activation of the matrix is limited to the colon. A platform incorporating such coating will enable activation of the liquid activated sensor only when the expandable platform 100 is physically within the colon, i.e., the erodible coating 204 is eroded in the colon. For example, this principle could be incorporated in an expandable platform 100 targeted for retention at the ileocecal valve. Only when the expandable platform 100 is emptied into the colon will an erodible coating 204 comprising a colonic erodible matrix erode and allow the liquid-activated sensor 206 to be activated, thus indicating that platform is out of the small intestine. The colonic erodible matrix may include materials that erode (degrade) when exposed to colonic microbiota. For example, a colonic erodible matrix can comprise certain polysaccharides such as chitosan, alginate, amylose, pectin, galactomannan, etc. An example of such a formulation, which utilizes an amylose coating, is disclosed in European Patent Publication No. 0343993. Alternatively, the erodible coating 204 can comprise a mixture of two or more materials incorporated in the colonic activated matrix, such as a pH dependent polymer and a polysaccharide.

In another embodiment, the erodible coating 204 can comprise a pH-dependent enteric polymer. An enteric polymer may be understood as a polymer that does not readily dissolve or degrade under the typical pH and other physical conditions of a human stomach, but that does dissolve or degrade at pH and other physical conditions of the intestinal tract of a human, i.e., the conditions that exist following passage from the stomach through the pylorus (i.e., pH>5). The enteric polymer may be a pH dependent polymer with a dissolution threshold between, e.g., pH 6.0 and pH 7.0. For example, an enteric polymer may be made from or include cellulose acetate phthalate, hydroxypropyl methyl-cellulose phthalate 50 and 55, or copolymers of methacrylic acid and methyl methacrylate (e.g., Eudragit® S 100, Eudragit® L, Eudragit® FS, and Eudragit® P4135 F).

In aspects where erodible coating 204 is sensitive to the pH of its environment, erodible coating 204 may refer to one pH dependent polymer, a mixture of two or more pH dependent polymers, or a mixture of polymers of which at least one is a pH dependent polymer, as long as the resulting mixture is pH dependent in nature.

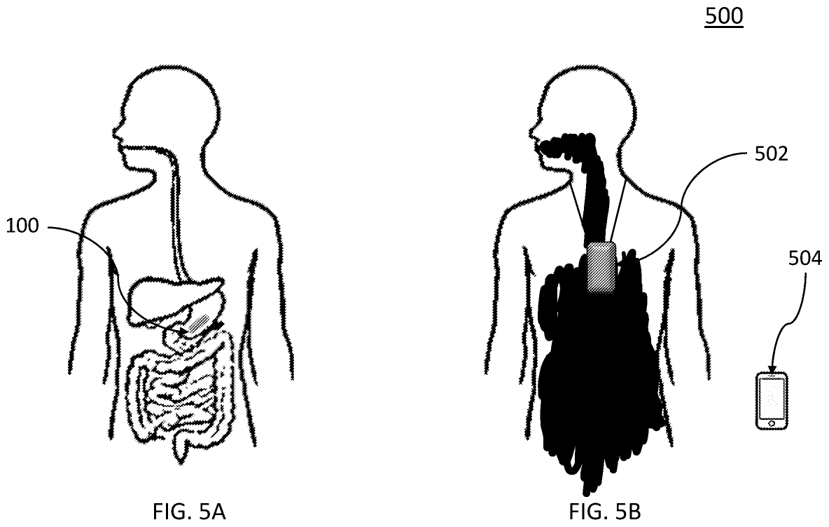

Emptying monitoring indicator 202 may interact with an external receiver or mobile device that detects a signal from liquid-activated sensor 206 after erodible coating 204 has eroded and liquid-activated sensor 206 has been activated through contact with a liquid. Such external receiver or mobile device is described in further detail with respect to FIG. 5B. In other aspects, and as illustrated in FIGS. 2C, 2D, and 3B, expandable platform 100′ can include an emptying monitoring indicator 202 comprising a magnet 208 coupled to at least one of the at least two articulating arms 102-n of the expandable platform, through one of the previously described couplings. As expandable platform 100′ shares common features with expandable platform 100, identical reference numbers will be used. Magnet 208 emits a detectable magnetic field. Magnet 208 may be coupled to one of the at least two articulating arms 102-n via press-fit, integrally molded or formed into the articulating arm 102-n, or coupled via coating 207, as will be described further below. Magnet 208 may be an electromagnetic coil that produces an AC magnetic field, or a permanent magnet. In an aspect where magnet 208 is an electromagnetic coil that produces an AC magnetic field, magnet 208 includes an energy source for the electromagnetic coil. The energy source may be a battery coupled to magnet 208, or another internal or external energy source (e.g., external energy source where the electromagnetic coil includes pickup coils). In this aspect, the magnetic field produced is approximately equal to a magnetic field produced by an ideal magnetic dipole. In an alternative aspect where magnet 208 is a permanent magnet, magnet 208 may be a rare earth magnet. In one embodiment, the magnet 208 is a neodymium magnet, though other compositions of magnets are contemplated. In this aspect, magnet 208 is coupled directly to at least one of the at least two articulating arms 102-n or its arm segment. Magnet 208 may be coupled directly to the at least one of the at least two articulating arms 102-n or its arm segment through the indicator cavity 213. In one embodiment, the magnet 208 is sealed within a coating 207. The coating 207 may comprise silicone, though other types of coatings are contemplated. Alternatively, the magnet 208 could have an outer surface that may allow magnet 208 to directly contact the GI tract environment. Emptying monitoring indicator 202 may interact with an external receiver or mobile device that detects the magnetic field emitted from magnet 208. Such external receiver or mobile device is described in further detail with respect to FIG. 5B.

In operation, the magnet 208 may be coupled to one of the at least two articulating arms 102-n such that the positive side of the magnet 208 faces outwards when the expandable platform 100′ is in the expanded configuration. This orientation would minimize the risk that in the rare event that two expandable platforms 100′ come into contact with each other within a gastric tract the two expandable platforms 100′ will attach to each other under force of the respective magnets 208.

Expandable platform 100 may transfer from the expanded configuration into the emptying configuration upon locking assembly 212 being deactivated. FIG. 4 is an illustration of the expandable platform 100 in an emptying configuration according to some aspects of the present disclosure. For example, in the case of locking assembly 212 comprising a locking member, the deactivation of locking assembly 212 may comprise or consist in detaching at least one end of the locking member from its respective coupling head 104, thereby releasing the constraint operated by the locking member. In any case where locking assembly 212 is deactivated, expandable platform 100 may be so configured that deformable member 106 (which is not shortened anymore) now forces again the coupling heads 104 together, but insufficiently to resist to GI forces. As a result, the device is said to be in its emptying configuration, as it is not capable of being retained at the predetermined location and may thus be emptied. Alternatively, the device may be configured such that, upon locking assembly 212 being deactivated, deformable member 106 is prevented from forcing the coupling heads 104 together at all, thereby allowing transfer into the emptying configuration. For example, the device may be configured such that, upon locking assembly 212 being deactivated, at least one end of deformable member 106 is detached from its respective coupling head 104. Further, at least one of the arms 102-n can be detached from the coupling heads 104 in the emptying configuration.

Expandable platform 100 may contain a component to cause expandable platform 100 to transfer from the expanded configuration into the emptying configuration, such as a trigger assembly. The trigger assembly may comprise one or more support elements configured to temporarily maintain expandable platform 100 in the expanded configuration. For example, one or more support elements may be configured to be disabled upon activation of the trigger assembly thereby causing expandable platform 100 to transfer into the emptying configuration. The support elements may have a pin shape and may be located inside coupling heads 104.

In some aspects, one or more support elements may be configured to be disabled when a surrounding pH reaches a predetermined pH threshold. Such one or more support elements may optionally be at least partially made of a pH dependent polymer configured to dissolve when the surrounding pH reaches the predetermined pH threshold. For example, the support elements may be made of time dependent polymers such as HPMC or of Eudragit® EPO that are soluble in the gastric milieu (low pH). In examples where the predetermined location is within the intestinal tract such as the ileocolic valve, one or more support elements may be at least partially made of a material configured to degrade at standard ileocolic environmental conditions. For example, one or more support elements comprise a material configured to degrade when the surrounding pH is between 6.5 and 7.5. Additionally or alternatively, one or more support elements may be at least partially made of a pH dependent polymer configured to dissolve when the surrounding pH reaches a predetermined pH threshold, preferably when the surrounding pH reaches below pH 5. In examples where the predetermined location is the stomach, one or more support elements may be at least partially made of a material configured to degrade at standard stomach environmental conditions. For example, the one or more support elements may comprise a material configured to degrade when the surrounding pH is between 1 and 3 or between 1 and 4 or 5. Additionally or alternatively, one or more (other) support elements may be at least partially made of a pH dependent polymer configured to dissolve when the surrounding pH reaches the predetermined pH threshold, preferably when the surrounding pH reaches above pH 6. In such cases, at least one of the support elements may be activated by dosing a tablet that alters the stomach pH so to disassemble and empty the device from the stomach (by transferring the device from the expanded configuration into the emptying configuration). Such treatments may be considered as a “safety mechanism”, e.g., in case emptying from stomach is required.

In alternative aspects, expandable platform 100 may be configured for the trigger assembly to self-activate after being exposed to the GI tract over a predetermined period of time.

In some aspects, the one or more support elements may be configured to form one or more structural weak points of expandable platform 100 to enable a collapse of expandable platform 100 into the emptying configuration. The one or more structural weak points may enable disassembly of the device into at least two disassembled subcomponents. This may contribute to improving a transfer from the expanded configuration into the emptying configuration.

FIGS. 5A-5B are illustrations of a system 500 for detecting information from an emptying monitoring indicator, according to some aspects of the present disclosure. FIG. 5A illustrates an internal view of a patient's body, while FIG. 5B illustrates an external view of a patient's body. FIG. 5A illustrates expandable platform 100, as described above, in a closed configuration within the stomach of a patient. FIG. 5B illustrates receiver 502 and mobile device 504, both external to the patient's body.

As described above, expandable platforms 100, 100′ include emptying monitoring indicators 202, 202′. FIGS. 2A, 2B, and 3A illustrate aspects where emptying monitoring indicator 202 includes liquid-activated sensor 206 and erodible coating 204, while FIGS. 2C, 2D, and 3B illustrate aspects where emptying monitoring indicator 202′ includes magnet 208. Aspects of system 500 are compatible with both types of emptying monitoring indicators 202, 202′.

In system 500, receiver 502 is positioned outside the body of the patient. Receiver 502 may be worn by the patient, such as around the neck or around the waist. Receiver 502 is configured to detect information from an internal device, such as expandable platform 100, 100′ for dispensing a medication and having emptying monitoring indicator 202, 202′. Receiver 502 is any device capable of detecting and sending signals, such as a device with an antenna, an amplifier, and associated filters to separate frequencies of detected signals.

In aspects where emptying monitoring indicator 202 includes liquid-activated sensor 206 and erodible coating 204, liquid-activated sensor 206 transmits a signal to receiver 502 when it is activated. As described above, liquid-activated sensor 206 may activate after erodible coating 204 has eroded and liquid-activated sensor 206 has contacted a liquid. The signal transmitted by liquid-activated sensor 206 and detected by receiver 502 may be a wireless communication signal, such as via a Bluetooth Low Energy (BLE) protocol or a near field communication (NFC) protocol. Thus, when receiver 502 detects a signal from liquid-activated sensor 206, it means that a retentive device, such as expandable platform 100, has dispensed medication and has exited the predetermined location of the GI tract. More specifically, it means that expandable platform 100 is in the emptying configuration and has proceeded to a portion of the GI tract of a certain pH, capable of eroding the erodible coating 204, indicating that expandable platform 100 is no longer at the predetermined location of the GI tract. As such, at this point it is permissible to ingest a second expandable platform that includes a second dose of medication. Alternatively, when receiver 502 has not detected a signal from liquid-activated sensor 206, it means that expandable platform 100 has not yet entered emptying configuration or has not yet proceeded past the predetermined location of the GI tract to a condition capable of eroding the erodible coating 204. As such, at this point it is not permissible to ingest a second expandable platform that includes a second dose of medication.

In aspects where emptying monitoring indicator 202′ includes magnet 208, magnet 208 emits a signal that comprises a magnetic field. Receiver 502 is configured to sense magnetic fields from magnet 208 through an array of sensors. The array of sensors may be inductive sensors, magnet-resistive sensors, flux-gate sensors, or any other type of sensor applicable to magnets. The receiver may continuously calculate a momentum and position of magnet 208 through the sensed magnetic field. In some aspects, the position of each magnet 208 may be defined by five coordinates (x, y, z, θ, φ) representing three translations (x, y, z) and two rotations (θ, φ). When receiver 502 senses a magnetic field from magnet 208, it indicates that magnet 208 is still within the body of the patient and/or within the predetermined location (e.g., in the expanded configuration). As such, at this point it is not permissible to ingest a second expandable platform that includes a second dose of medication. Alternatively, when receiver 502 cannot sense a magnetic field from magnet 208, it indicates that magnet 208 is outside the body of the patient and/or outside the predetermined location. As such, at this point it is permissible to ingest a second expandable platform that includes a second dose of medication. This may be due to magnet 208 being outside the sensing proximity of receiver 502.

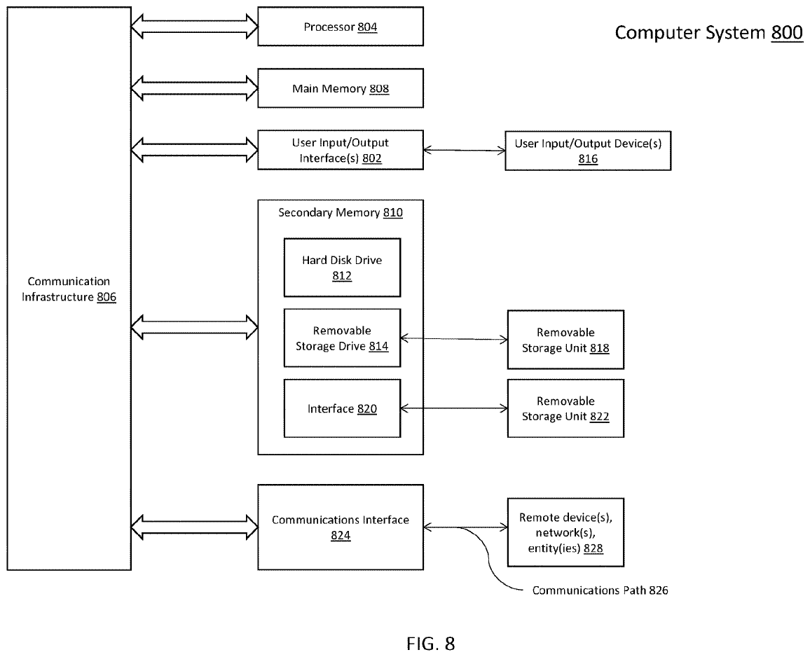

After receiver 502 has detected a signal from either of the above described aspects of emptying monitoring indicator 202, 202′, receiver 502 is configured to transmit a signal to mobile device 504. Receiver 502 may transmit the signal to mobile device 504 via any form of wireless communication, such as Bluetooth, Local Area Networks, Wide Area Networks, the Internet, etc. Receiver 502 may also transmit the signal to mobile device 504 via any form of wired communication. Receiver 502 may process the signal or magnetic field from emptying monitoring indicator 202 before transmitting the signal to mobile device 504. Receiver 502 may process the signal or magnetic field via a processor and other components as provided by FIG. 8. In some aspects, receiver 502 may process the signal or magnetic field using algorithms capable of providing location information.

Mobile device 504 may be any mobile device capable of wireless communication, such as through Bluetooth, Local Area Networks, Wide Area Networks, the Internet, etc. Such devices may include a smartphone, laptop, tablet, smart watch, etc. Mobile device 504 may also be capable of wired communication. Mobile device 504 is configured to receive a signal from receiver 502 that is based on the signal received from the emptying monitoring indicator 202, 202′ and is indicative of whether expandable platform 100, 100′ has emptied from a predetermined location of the GI tract. Mobile device 504 may either process the signal from receiver 502 and generate patient instructions from the signal, or may generate patient instructions from a pre-processed signal. If the signal indicates that expandable platform 100 remains at the predetermined location of the GI tract, or in the case of magnet 208, that it remains in the body of the patient, then mobile device 504 may generate patient instructions instructing the patient that it is not safe to take another dose of medication via a retentive device. Alternatively, if the signal indicates that expandable platform 100, 100′ has emptied from the predetermined location of the GI tract, or in the case of magnet 208, that it has exited the body of the patient, then mobile device 504 may generate patient instructions instructing the patient that it is safe to take another dose of medication via a retentive device. The patient instructions may be in the form of an audio, haptic, and/or text alert, message, or communication provided by the mobile device 504.

In some aspects, receiver 502 and/or its functionality is incorporated into mobile device 504, so that a separate receiver is not required. In some aspects, mobile device 504 may receive signals directly from expandable platform 100.

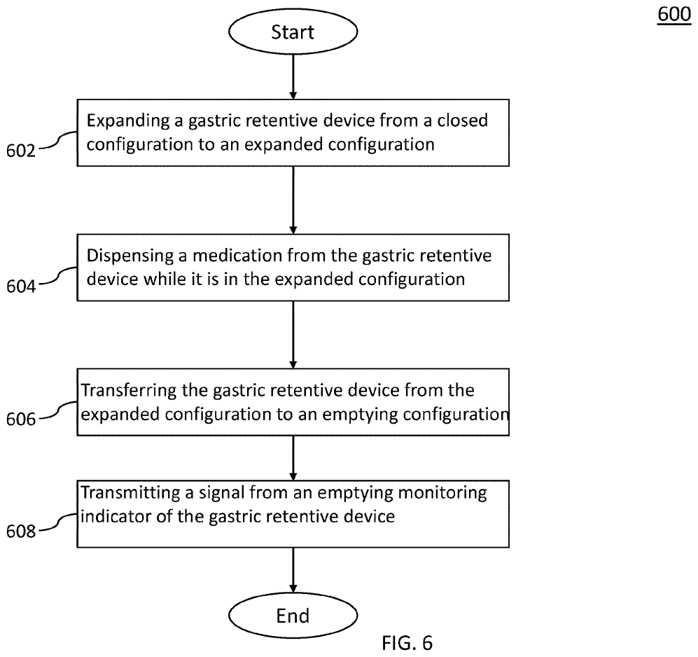

FIG. 6 is a flowchart of a method 600 for monitoring emptying of a retentive device from a predetermined location of a GI tract, according to an aspect of the invention. It is to be appreciated that not all steps may be needed to perform the disclosure provided herein. Further, some of the steps may be performed simultaneously, or in a different order than shown in FIG. 6, as will be understood by a person of ordinary skill in the art.

In step 602, an expandable platform 100 for dispensing medication to a predetermined location in the GI tract is expanded from a closed configuration to an expanded configuration. The expandable platform 100, 100′ may include at least two articulating arms 102-n, a first and a second coupling head 104, a deformable member 106, a locking assembly 212, a medication cavity 210, a medication 211, and various other components that work together to dispense the medication 211 and to change the configuration of the device. The expandable platform may be configured to transfer among various configurations. These configurations may include closed, expanded, and emptying configurations as described with respect to expandable platform 100. The closed configuration may be the state of the retentive device prior to administration where the retentive device has a size that is suitable for swallowing or for oral manipulation with an endoscope. The expanded configuration may be the state when the retentive device resides at the predetermined location. When the retentive device is in the expanded configuration and positioned at the predetermined location of the GI tract, the retentive device may be configured to be retained at the predetermined location of the gastrointestinal tract. The predetermined location may be the stomach, such as at the pyloric valve, or any location within the small intestine, such as at the ileocecal valve, or any location within the large intestine.

In step 604, a medication is dispensed from the expandable platform while it is in the expanded configuration. For example, the expandable platform may dispense medication at a predetermined location of the GI tract through temporary residence at the predetermined location. The retentive device, such as expandable platform 100, may have a medication cavity that is only opened and/or exposed when the retentive device is in the expanded configuration. In some aspects, the medication is dispensed immediately or soon after expansion. In some aspects, the medication is dispensed slowly over a period of time after expansion.

In step 606, the retentive device is transferred from the expanded configuration to an emptying configuration. In aspects, the transfer from the expanded configuration into the emptying configuration is irreversible. The emptying configuration may be, for example, a disassembled configuration.

In step 608, a signal is transmitted from an emptying monitoring indicator of the gastric retentive device. In one embodiment, step 608 may occur after the expandable platform 100 transfers to the emptying configuration. As described with respect to expandable platform 100, for example, the retentive device may have an emptying monitoring indicator that is coupled to the expandable platform. In some aspects, the emptying monitoring indicator may be or include a liquid-activated sensor coupled to at least one of the at least two articulating arms of the expandable platform. The liquid-activated sensor may be a liquid level switch that is designed to send a signal when immersed in a liquid, such as water or chyme fluid. The liquid-activated sensor may be an optical sensor, a capacitive sensor, a conductive sensor, a diaphragm sensor, a float sensor, or any other type of liquid-activated sensor that can be coupled to the expandable platform. The liquid-activated sensor may be enclosed by an erodible coating, temporarily protecting it from certain conditions of the GI tract. The erodible coating may erode based on conditions (e.g., pH) of the organs of the GI tract, thus causing transmission of the signal once liquid comes in contact with the exposed sensor. The erodible coating may be a pH dependent polymer, whose degradability or dissolubility changes depending on the pH of a surrounding solution. The erodible coating may refer to one or more of a pH dependent polymer, a mixture of two or more pH dependent polymers, or a mixture of polymers of which at least one is a pH dependent polymer, as long as the resulting mixture is pH dependent in nature. The erodible coating may be a colonic enzymatic dependent polymer, whose degradability or dissolubility changes by microbiome fermentation in surrounding colonic solution.

In other aspects, the emptying monitoring indicator may be or include a magnet coupled to at least one of the at least two articulating arms of the expandable platform. In this embodiment, step 608 may occur between steps 604 and 606. The magnet may emit a detectable magnetic field, as described above with respect to magnet 208. As such, detection of the magnet 208 indicates the expandable platform 100′ is still within the gastric tract, while failure to detect the magnet indicates that the expandable platform 100′ is in the emptying configuration and has passed through the gastric tract.

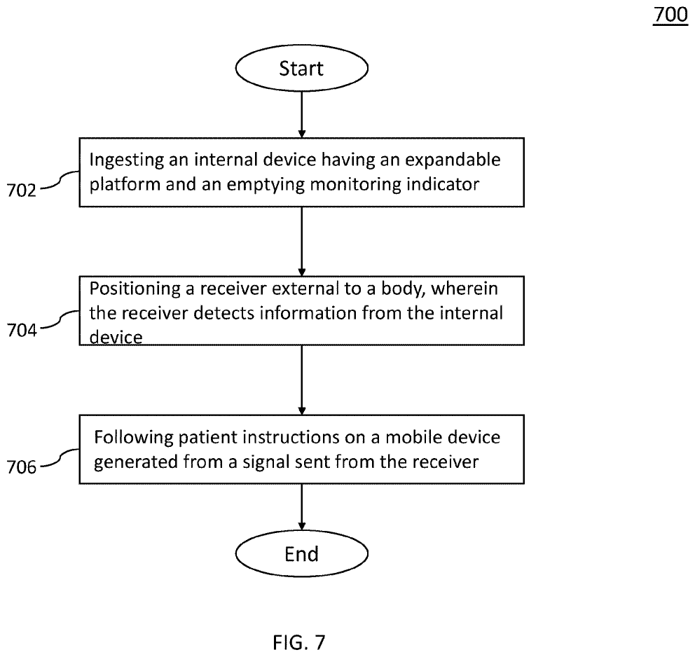

FIG. 7 is a flowchart of a method 700 for monitoring emptying of a retentive device from a predetermined location of a GI tract, according to an aspect of the invention. It is to be appreciated that not all steps may be needed to perform the disclosure provided herein. Further, some of the steps may be performed simultaneously, or in a different order than shown in FIG. 7, as will be understood by a person of ordinary skill in the art.

In step 702, a retentive device is ingested. The retentive device may be configured to dispense a medication at a predetermined location of the GI tract through temporary residence at the predetermined location. The predetermined location may be the stomach or any location within the small intestine or the large intestine.

The retentive device may have an expandable platform for dispensing a medication. The expandable platform may include at least two articulating arms, a first and a second coupling head, a deformable member, a locking assembly, a medication cavity, an emptying monitoring indicator, and various other components that work together to dispense the medication and to change the configuration of the device, such as expandable platform 100 described above. The retentive device may be configured to transfer among various configurations. These configurations may include closed, expanded, and emptying configurations as described above.

In step 704, a receiver is positioned external to a body of a patient. The receiver may be worn by the patient, such as around the neck or around the waist. The receiver is configured to detect information from the emptying monitoring indicator of the retentive device, ingested in step 702.

In step 706, patient instructions are followed on a mobile device. The patient instructions are generated from a signal sent from the receiver in step 704, based on the information detected by the receiver in step 704. In some aspects, the patient instructions instruct a patient whether it is safe to take another dose of the medication. In some aspects, the patient instructions identify whether the retentive device is still located within the body and/or the location of the retentive device in the body.

Various aspects can be implemented, for example, using one or more computer systems, such as computer system 800 shown in FIG. 8. Computer system 800 can be used, for example, in receiver 502 or mobile device 504. For example, computer system 800 can detect information from an internal device having an expandable platform for delivering a medication and having an emptying monitoring indicator coupled to the expandable platform, such as expandable platform 100 described above. Computer system 800 can receive a signal from a receiver based on information detected from an internal device, according to some aspects. Computer system 800 can also generate patient instructions from a signal received from a receiver. Computer system 800 can be any computer capable of performing the functions described herein.

Computer system 800 can be any well-known computer capable of performing the functions described herein.

Computer system 800 includes one or more processors (also called central processing units, or CPUs), such as a processor 804. Processor 804 is connected to a communication infrastructure 806 (e.g., a bus).

One or more processors 804 may each be a graphics processing unit (GPU). In an aspect, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 800 also includes user input/output device(s) 816, such as monitors, keyboards, pointing devices, etc., that communicate with communication infrastructure 806 through user input/output interface(s) 802.

Computer system 800 also includes a main or primary memory 808, such as random access memory (RAM). Main memory 808 may include one or more levels of cache. Main memory 808 has stored therein control logic (i.e., computer software) and/or data.

Computer system 800 may also include one or more secondary storage devices or secondary memory 810. Secondary memory 810 may include, for example, a hard disk drive 812 and/or a removable storage drive 814 or device. Removable storage drive 814 may be a floppy disk drive, a magnetic tape drive, a compact disk drive, an optical storage device, tape backup device, and/or any other storage device/drive.

Removable storage drive 814 may interact with a removable storage unit 818. Removable storage unit 818 includes a computer usable or readable storage device having stored thereon computer software (control logic) and/or data. Removable storage unit 818 may be a floppy disk, magnetic tape, compact disk, DVD, optical storage disk, and/any other computer data storage device. Removable storage drive 814 reads from and/or writes to removable storage unit 818 in a well-known manner.

According to an exemplary aspect, secondary memory 810 may include other means, instrumentalities or other approaches for allowing computer programs and/or other instructions and/or data to be accessed by computer system 800. Such means, instrumentalities or other approaches may include, for example, a removable storage unit 822 and an interface 820. Examples of the removable storage unit 822 and the interface 820 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 800 may further include a communication interface 824. Communication interface 824 enables computer system 800 to communicate and interact with any combination of remote devices, remote networks, remote entities, etc. (individually and collectively referenced by reference number 7828). For example, communication interface 824 may allow computer system 800 to communicate with remote devices 828 over communications path 826, which may be wired and/or wireless, and which may include any combination of LANs, WANs, the Internet, etc. Control logic and/or data may be transmitted to and from computer system 800 via communication path 826.

In an aspect, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 800, main memory 808, secondary memory 810, and removable storage units 818 and 822, as well as tangible articles of manufacture embodying any combination of the foregoing. Such control logic, when executed by one or more data processing devices (such as computer system 800), causes such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 8. In particular, aspects can operate with software, hardware, and/or operating system implementations other than those described herein.

It is to be appreciated that the Detailed Description section, and not any other section, is intended to be used to interpret the claims. Other sections can set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible, and are within the scope and spirit of this disclosure. For example, and without limiting the generality of this paragraph, aspects are not limited to the software, hardware, firmware, and/or entities illustrated in the figures and/or described herein. Further, aspects (whether or not explicitly described herein) have significant utility to fields and applications beyond the examples described herein.

Aspects have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative aspects can perform functional blocks, steps, operations, methods, etc. using orderings different than those described herein.

References herein to “one aspect,” “an aspect,” “an example aspect,” or similar phrases, indicate that the aspect described can include a particular feature, structure, or characteristic, but every aspect can not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein.

The breadth and scope of this disclosure should not be limited by any of the above-described exemplary aspects, but should be defined only in accordance with the following claims and their equivalents.

What is claimed is:

1. An expandable platform for residence at a predetermined location of a gastrointestinal tract and dispensing a medication at the predetermined location, the expandable platform configured to transition between a plurality of configurations including a closed configuration, an expanded configuration, and an emptying configuration, the expandable platform comprising:
a deformable member configured to expand the expandable platform from the closed configuration to the expanded configuration, wherein the expandable platform is configured to reside at the predetermined location and dispense the medication when the expandable platform is in the expanded configuration;
a locking assembly configured to maintain the expandable platform in the expanded configuration for a predetermined time, when the predetermined time expires the locking assembly is configured to release the expandable platform and the expandable platform is configured to transition from the expanded configuration into the emptying configuration, in which the expandable platform is capable of passing through the predetermined location of the gastrointestinal tract; and
an emptying monitoring indicator configured to wirelessly transmit a signal indicative of at least one of a configuration of the plurality of configurations or a location of the expandable platform.

2. The expandable platform of claim 1, wherein the expandable platform comprises at least two articulating arms, wherein the emptying monitoring indicator comprises:
a liquid-activated sensor coupled to at least one of the at least two articulating arms of the expandable platform, wherein the liquid-activated sensor has an outer surface; and
an erodible coating that encloses the outer surface of the liquid-activated sensor,
wherein when the erodible coating erodes to expose the outer surface of the liquid-activated sensor to the gastrointestinal tract, the liquid-activated sensor transmits the signal.

3. The expandable platform of claim 2, wherein the liquid-activated sensor transmits the signal when the outer surface is exposed to water or chyme fluid.

4. The expandable platform of claim 2, wherein the erodible coating is a colonic erodible matrix.

5. The expandable platform of claim 2, wherein the erodible coating is a pH-dependent polymer with a dissolution threshold between pH 6.0 and pH 7.0.

6. The expandable platform of claim 2, wherein the erodible coating is an enteric coating polymer.

7. The expandable platform of claim 2, wherein detection of the signal by a receiver external to the gastrointestinal tract indicates that the expandable platform has passed through the predetermined location.

8. The expandable platform of claim 1, wherein the emptying monitoring indicator comprises a magnet, the signal comprising a magnetic field, wherein detection of the signal by a receiver external to the gastrointestinal tract is indicative of the expandable platform being within the gastrointestinal tract.

9. The expandable platform of claim 1, further comprising:
at least two articulating arms, wherein each of the at least two articulating arms has a first end and a second end; and
a first and a second coupling head, wherein the first end of each of the at least two articulating arms is coupled to the first coupling head and the second end of each of the at least two articulating arms is coupled to the second coupling head,
wherein the deformable member is configured to draw the first and the second coupling heads towards each other to expand the expandable platform from the closed configuration to the expanded configuration.

10. The expandable platform of claim 9, wherein at least one of the at least two articulating arms defines a medication cavity configured to receive the medication.

11. The expandable platform of claim 9, wherein the emptying monitoring indicator is coupled to at least one of the at least two articulating arms.

12. The expandable platform of any of the previous claims, wherein the detection of the signal by a receiver external to the gastrointestinal tract indicates whether a patient may take another dose of the medication.

13. A system for dispensing medication at a predetermined location of the gastrointestinal tract, comprising:
an expandable platform comprising a first dose of the medication and an emptying monitoring indicator configured to wirelessly transmit a first signal, the expandable platform configured to transition between a plurality of configurations including a closed configuration, an expanded configuration, and an emptying configuration, wherein the expandable platform is configured to be held at the predetermined location and dispense the medication in the expanded configuration and is configured to pass through the predetermined location in the emptying configuration; and
a receiver external to the gastrointestinal tract, the receiver configured to receive the first signal from the emptying monitoring indicator, the first signal being indicative of at least one of a configuration of the plurality of configurations or a location of the expandable platform.

14. The system of claim 13, further comprising:
a mobile device configured to receive a second signal from the receiver based on the first signal, the mobile device comprising a processor configured to determine whether a patient may take a second dose of the medication based on the second signal.

15. The system of claim 13, wherein:
the emptying monitoring indicator comprises:
a liquid-activated sensor comprising an outer surface; and
an erodible coating that encloses the outer surface of the liquid-activated sensor, and
wherein the liquid-activated sensor is configured to transmit the first signal when the erodible coating erodes to expose the outer surface of the liquid-activated sensor to the gastrointestinal tract.

16. The system of claim 14, wherein the liquid-activated sensor is configured to transmit the first signal when the expandable platform is in the emptying configuration,

17. The system of claim 16, wherein the receiver receives the first signal when the expandable platform has passed through the predetermined location.

18. The system of claim 13, wherein:
the emptying monitoring indicator comprises a magnet coupled to the expandable platform, wherein the first signal comprises a magnetic field emitted by the magnet when the expandable platform is in the gastrointestinal tract.

19. The system of claim 13, further comprising:
a mobile device comprising a processor and the receiver, wherein the processor is configured to determine whether a patient may take a second dose of the medication based on the second signal.

20. The system of claim 14 or 19, wherein the mobile device is configured to produce patient instructions indicating that the expandable platform is in the emptying configuration or has passed through the predetermined location.
